(19) United States
(12) Patent Application Publication
(10) Pub. No.: US 2013/0280779 A1
(43) Pub. Date: Oct. 24, 2013

(54) METHOD AND PLANT FOR THE METHANATION OF BIOMASS

(71) Applicant: SCHMACK BIOGAS GMBH, Schwandorf (DE)

(72) Inventors: Carsten HEMPEL, Kollmar (DE); Oliver JAROSCHEK, Munchen (DE)

(21) Appl. No.: 13/918,619

(22) Filed: Jun. 14, 2013

Related U.S. Application Data
(63) Continuation of application No. PCT/EP2011/006238, filed on Dec. 10, 2011.

(30) Foreign Application Priority Data
Dec. 15, 2010 (DE) 102010054676.3

(51) Int. Cl. C12P 5/02

(52) U.S. Cl. CPC C12P 5/023
USPC 435/167; 435/294.1

(57) ABSTRACT

A method for the methanation of biomass and the energetic use of the biogas obtained in a plant featuring at least one fermenter operating in a batch process, the fermenter is filled with biomass, a loading opening is closed in an airtight manner, methane-rich biogas produced in a fermentation phase is fed into a first gas storage unit, at least intermittently, during the flushing of the fermenter with flushing air, mixed gas with low methane content is fed into a second gas storage unit, at least intermittently, the loading opening is opened and the fermentation residue is removed from the fermenter. The energetic use of gas stored in the second gas storage unit is carried out such that gas removed from this gas storage unit is mixed with the biogas removed from the first gas storage unit before the mixed gas produced in this way is subsequently used energetically.

### CROSS-REFERENCE TO RELATED APPLICATIONS

This is a continuation of PCT/EP2011/006238, filed Dec. 10, 2011, which claims priority to DE Application 10 2010 054 676.3 filed Dec. 15, 2010, the contents of each of which are incorporated by reference in their entireties.

### FIELD OF THE INVENTION

The present invention relates to a process for the methanation of biomass and energetic use of the biogas obtained in a plant that features at least one fermenter operating in batch processing mode. In addition, the invention relates to a plant suitable for executing such a process for the methanation of biomass and energetic use of the biogas produced.

### BACKGROUND

Processes for the methanation of biomass and energetic use of the biogas produced as well as of plants suitable to execute such processes are known in different variations. There are a number of distinct concepts, which, for example, differ with regard to the consistency of the biomass to be methanized (a pumpable substrate on the one hand, or a pourable substrate with relatively high dry matter content on the other), or with regard to process management (continuous-flow fermenters on the one hand, or discontinuously operated batch processing fermenters on the other).

Preferred under various conditions, one type of plant is specifically geared toward the discontinuous batch fermentation process of a pourable substrate with relatively high dry matter content. Relevant practical aspects that come into effect here are a comparably high flexibility with regard to the methanizable biomass, meaning the raw material, and good process control management—even with rather non-homogenous raw material. Pertinent prior art with regard to this type of plant include DE 10257849 A1, DE 19719323 A1, DE 10050623 B4, EP 934998 B1, DE 10034279 A1, WO 02/06439 A2, EP 1681274 A2, DE 102008015240 A1 and EP 1997875 A1.

U.S. Pat. No. 7,211,429 B2 also presents a process of the previously mentioned type to methanize biomass, as well as a plant suitable to execute the respective process. The latter preferably includes several fermenters, operated in a phase-shifted manner and utilizing a two- or three-step process for breaking down organic matter. A (anaerobic) methanation phase is preceded by a rotting phase, during which the biomass is aerobically composted, producing mostly CO2, where the heat resulting from this process raises the temperature of the biomass, which in turn has a favorable effect on the subsequent fermentation. As an option after completion of the methanation, further composting takes place, with the fully fermented substrate being aerated for this purpose. The individual fermenters of the plant are connected with each other, so that, for example, one fermenter can be inoculated with leachate extracted from another fermenter operating in a phase-shifted mode in order to stimulate fermentation, or to allow for the use of the warm exhaust air from a fermenter operating in the aerobic phase to raise the biomass temperature of another fermenter in order to stimulate methanation in the latter.

While the operational reliability and dependability of the known plants and processes are quite satisfying to date, there is still a quest for ways to increase economic efficiency. The ecologically desirable dissemination of technology for methanation of biomass and energetic use of the biogas produced can be expected only if processes and plants are made available that operate more efficiently than hitherto, without any loss of environmental compatibility.

### SUMMARY

Against this background, the present invention has set itself the task to make available a particularly efficient and, at the same time, environmentally friendly process for the methanation of biomass and the energetic use of the biogas produced in a unit featuring at least one fermenter operating in batch processing mode and a plant suitable to execute the respective process.

According to the present invention, as indicated in claim 1, this task is solved by implementing a process for the methanation of biomass and the energetic use of the biogas produced in a plant featuring at least one fermenter operating in batch processing mode, including the following sequence of steps which is cyclically repeated in the at least one fermenter:
a) Filling the fermenter with biomass through a loading hatch open to the environment,
b) airtight closing of the loading hatch,
c) feeding methane-rich biogas produced during the fermentation phase, at least intermittently, into a first gas storage unit,
d) flushing the fermenter with flushing air, and, at least intermittently, feeding the mixed gas with low methane content, which leaves the fermenter during the flushing phase, into a second gas storage unit,
e) opening the loading hatch, and
f) removal the digestate from the fermenter, wherein gas is removed from the second gas storage unit and is mixed with biogas removed from the first gas storage unit, and the mixed gas produced in this way is subsequently used energetically.

In functional interaction with the other features, as they are determining factors for the process according to the invention, of particular importance for the latter is that the gas, which leaves the particular fermenter with flushing air through the gas outlet during flushing, is fed for at least one time period during the flushing phase into a second gas storage unit, which is separate from the (first) gas storage unit, into which, at least during part of the fermentation phase, the methane-rich biogas (for example, CH, content>48%) leaving the particular fermenter's gas outlet is fed, Furthermore, the gas that is taken from the second gas storage unit is mixed with the biogas taken from the first gas storage unit, and the biogas produced is subsequently used energetically. This allows for a surprisingly simple way of achieving several positive effects with regard to economic efficiency. On the one hand, the mixed gas with comparably low methane content, which leaves the fermenter during the flushing phase and whose low methane concentration conflicts with efficient energetic utilization, can be used in the plant provided for energetic utilization of “normal,” meaning methane-rich biogas. This is achieved by collecting this mixed gas in a second gas storage unit, separate from the methane-rich biogas produced during the fermentation phase, and later using it energetically, where the gas removed from the second gas storage unit for energetic utilization is mixed with the methane-rich gas, which is removed from the first gas storage unit at the same time, resulting in a gas mixture to be used energetically. In this way, it is possible to increase the content of energetically used biogas, which positively affects economic efficiency and environmental compatibility because less gas is released into the environment via a torch, a filter or other units. With regard to economic efficiency, it is also advantageous that, compared to state-of-the art technology, the duration of the flushing phase can be significantly shortened, which, in other words, increases the productivity of a particular fermenter—and that of the plant as a whole—by reducing the fermenter's unproductive time. In this way, plants implementing the process according to the invention are able to achieve a higher throughput of biomass, while otherwise using state-of-the-art technology. Furthermore, economic efficiency is positively affected by storing two gases of different qualities in the first and second gas storage unit, which makes it possible to specifically influence the quality of the gas that is fed into the plant for the energetic use of the biogas. Under the framework of the present invention, only methane-rich biogas removed from the first gas storage unit can optionally be used energetically in the appropriate plant during select time periods, and during other time periods, the previously described gas mixture, which is created by mixing gas removed from the second gas storage unit with methane-rich biogas removed from the first storage unit, can be used energetically. The specific influence on the quality of the gas that is fed into the plant for energetic use of the biogas—made possible by the application of the present invention—namely in the form of gas quality standardization or adjustments based on current production requirements as well the processing characteristics of the particular plant, has a positive effect on the efficiency and longevity of the thermal power station or other units (i.e. gas motor) intended for energetic biogas use. Another preferred form of energetic use of the biogas produced by the process according to the invention is the feeding the gas into the natural gas network, in which case, given process implementation according to the invention, the gas distribution station or the gas supply station become the plants for energetic bigas use with regard to the presented technology. Achieving the quality of natural gas is the prerequisite for feeding gas into the natural gas network, though, which means that the biogas produced has to be treated before the feed-in, using known state-of-the art techniques for biogas processing (i.e., desulphurization, drying, CO₂removal).

Based on the connections presented above, it is evident that the first gas storage unit as well as the second gas storage unit only exist for the purpose of intermediate storage of the gas removed from the fermenter, or fermenters, in order to enable the subsequent energetic use of the gas stored in the second gas storage unit by mixing it with gas removed from the first gas storage unit. In particular, unlike inside the fermenters, no biological processes of any considerable extent occur inside the two gas storage units, which would substantially change the composition of the gas inside a particular gas storage unit. In particular, the fermenters themselves, therefore, do not represent gas storage units within the framework of the present invention.

In order to execute the process according to the invention explained above, the plant according to the invention for methanation of biomass and energetic use of the biogas produced, as indicated in claim 14, features at least one loading hatch which can be closed in an airtight manner; at least one gas outlet, as well as a fermenter featuring a flushing air intake; at least one flushing air blower; a first gas storage unit; a second gas storage unit; one gas line network with several gas regulating valves that connects at least one fermenter with the first and the second gas storage unit; a unit for energetic biogas use; and a system control unit that adjusts the gas regulating valves, where the gas regulating valves can be adjusted in such a way that by using at least one gas outlet, either the first gas storage unit or the second gas storage unit, or only the first gas storage unit or simultaneously the first as well as the second gas storage unit can be connected via flow technology to the plant for energetic biogas use.

Given a specific set of conditions, according a first preferred further development of the process according to the invention, the mixed gas leaving the fermenter can be fed into the first gas storage unit during an initial period of flushing. During this first period of the flushing phase and under typical conditions, the mixed gas leaving the fermenter contains often still a relatively high methane content (for example, a CH₄ content between 20% and 48%), so that it is comparable in usability to the methane-rich biogas obtained during the fermentation process. And even in the case of a significantly lower methane concentration (down to about 20%), feeding the still relatively methane-rich gas mix (CH₄ content, for example, >20%), which leaves the fermenter through the gas outlet during the first flushing phase into the first gas storage unit is not detrimental to the usability of the methane-rich biogas stored there—due to the relative volumes of methane-rich biogas produced during fermentation and of the mixed gas produced during flushing the fermenter with flushing air, which typically develop during methanation.

Per another preferred further development of the process according to the invention, the mixed gas with very low methane content (CH₄ content, for example, <20%)leaving the fermenter during the last period of flushing, is released into the environment through a biofilter. By not feeding this gas with extremely low methane content into the second gas storage unit, the energetic usability of the gas stored there is preserved. In addition, this creates advantages in terms of safety to be discussed below. Any biogas being produced, or mixed gas that falls into the lower levels of lean gas (for example, CH₄ content between 4% and 10%) can be burned using a torch (with auxiliary firing), as needed; in case of a methane content of more than 10%, burning using a torch without auxiliary firing can be considered, as needed.

Using the previously noted typical values to classify the biogas produced or the mixed gas produced (during flushing) as methane-rich biogas (CH₄ content, for example, >48%), relatively methane-rich gas (CH₄ content, for example, between 20% and 48%), gas with relatively low methane content or low calorific gas (CH₄ content, for example, between 4% and 20%), and gas with very low methane content or rest gas (CH₄ content, for example, <4%)—and implementing the process according the invention presented—the mixed gas leaving the fermenter, as the latter is being flushed with flushing air, is typically fed into the second gas storage unit if its methane content is, for example, between 4% and 20%, under certain circumstances also if it is relatively high-methane biogas with a methane content, for example, between 20% and 40%. Therefore, a plant designated for the execution of such a process is typically equipped with a unit that monitors the quality (i.e. the methane content) of the gas leaving a particular fermenter, where the respective unit has an effect on the system control unit by allowing the latter to operate the gas regulating valves depending on the particular gas quality. As an alternative with regard to system control, though—instead of continuous monitoring of gas quality dependent on the corresponding measured values—pure timing control is a consideration, where empirical values regarding the duration of individual phases and phase periods within a cycle are used for the corresponding timing programming of the system.

Within the framework of the present invention, the production and energetic use of the mixed gas preferably occurs intermittently. Especially preferred is the production and energetic use of mixed gas during the exchange of biomass (steps f and a) and during the first time period of the subsequent fermentation start-up phase, where the energetic use of the mixed gas, under certain circumstances, can already be initiated towards the end of the flushing phase. In this case, it is of particular advantage to mostly empty the second gas storage unit during the fermentation start-up phase.

Under specific conditions, per yet another preferred further development of the process according to the invention, the gas with typically relatively low methane content, which is produced during a first phase of the fermentation process (fermentation start-up phase) after closing the loading hatch, can be fed into the second gas storage unit. This allows for a higher sustainable quality level of the methane gas, as opposed to feeding all of the biogas into the first gas storage unit from the beginning of the fermentation phase onward. Depending on the specific biomass, it is possible under certain circumstances during the fermentation start-up phase for a biogas to develop with such high methane concentration that a feeding of the biogas leaving the fermenter during this phase into the first gas storage unit is appropriate.

Typically, when applying the process according to the invention, the feed-in phases of mixed gas with a relatively low methane content into the second gas storage unit during the flushing phase are much shorter than the phases of removing gas from the second gas storage unit for its energetic use. In other words, the energetic use of the gases stored in the second gas storage unit typically extends over a longer period of time, compared to the duration of feeding the mixed gas with relatively low methane content into the second gas storage unit during the flushing phase. At this point, it becomes important that during the application of the present invention, the flushing process can be designed to be comparatively short in order to support high productivity, as already explained above. Under certain circumstances, then, the flushing process in typical dimensional fermenters (i.e. 1.000 m³) can be shortened to a few hours. In this respect, another preferred further development of the process according to the invention is distinguished by the fact that the duration of the flushing phase is only 0.2 to 1.0%, especially preferred even only 0.3 to 0.8% of the entire cycle duration. In terms of the speed of the gas exchange inside the fermenter during the flushing phase, the especially short flushing phase—made possible by applying the present invention—can also be expressed by noting that the rate of gas exchange during the flushing of the fermenter preferably is between 1/h and 10/h, especially preferred between 2/h and 6/h, with regard to the gas volume inside the fermenter.

Considering safety aspects, it is especially advantageous—according to yet another preferred further development of the process according to the invention—to always keep the methane concentration in the second gas storage unit above 16.5%, and thereby always above the explosion limit for methane in air, by adding methane-rich biogas. Given that because of the feed-in of mixed gas leaving the fermenter during the phase in which the latter is being flushed with flushing air, air also reaches the second gas storage unit. The risk of a gas explosion is minimized by applying the present further development by feeding methane-rich biogas into the second gas storage unit, preferably before the feed-in of mixed gas with relatively low methane content from the flushing phase, so that the methane concentration of the gas present in the second gas storage unit is always kept above the upper explosion limit in air.

Methane-rich biogas is particularly well suited for the present “enrichment” of the gas stored in the second gas storage unit, which—under a process management in which pourable biomass is methanized by irrigation with a percolate removed from a percolate fermenter—is being removed from the percolate fermenter. In this application of the invention, just this methane-rich biogas, removed from the percolate fermenter, is being fed into the second gas storage unit as a base gas, before the mixed gas with low methane content from the flushing phase is fed into the second gas storage unit.

However, it must be noted that it is not necessary to feed the methane-rich biogas as the aforementioned base gas into the second gas storage unit. Rather, methane-rich biogas produced in the fermenter during the fermentation phase is essentially also well suited in this case. In that sense, methane-rich biogas from the fermenter can also be fed into the second gas storage unit during the fermentation phase. In addition, considering that for the enrichment of the gas stored in the second gas storage unit, methane-rich biogas is being removed from the first gas storage unit and fed into the second gas storage unit. In that sense, the process according to the invention is completely independent on (methane-rich) biogas produced in a percolate fermenter; consequently it can run particularly in plants that do not feature a percolate fermenter, or in plants where the biogas produced in a percolate fermenter is being used in a different manner.

In order to clarify the terminology, it must be noted that “flushing” should under no circumstances be interpreted as the exchange of gas existing above the fermentation substrate with air. Rather, the flushing also includes the active ending of the remaining methane production by exposing the (anaerobic) methane-producing bacteria to an aerobic atmosphere, where a certain amount of new methane is still being produced during the flushing process as well. Insofar, the flushing is still a part of the biological processes. One of the distinguishing aspects of the present invention, as discussed, is that due to the specific treatment of the mixed gas leaving the fermenter during the flushing phase, this part of the process is accelerated, which in turn allows for a shortening of the flushing phase.

For clarification alone, it is noted that all data shown in percent are to be interpreted as % percent by volume. Furthermore, it is clarified that in a plant with several fermenters, the individual fermenters are typically being operated in a phase-shifted manner; insofar, specifications regarding a particular phase of a cycle running inside a fermenter refer to the respective fermenter. It is finally pointed out that the specification indicating that at least one fermenter features a gas outlet may under no circumstances be interpreted in such a way that only one gas outlet per fermenter is being provided for, which can optionally be connected to the first or the second gas storage unit. To the contrary, it is possible for each fermenter's gas line network to be made up of two gas lines, where biogas is fed through one of the two gas lines into the first gas storage unit, and gas is fed through the other gas line into the second gas storage unit, and each one of the two gas lines is connected via its own opening to the respective fermenter. This is particularly useful when, as is typically the case (see above), the mixed gas produced during the flushing phase is fed into the second gas storage unit at a much higher volume flow rate than the methane-rich biogas fed into the first gas storage unit during the fermentation phase; because in this case, the line diameters can be adapted according to the significantly different throughputs. Furthermore, the gas line network can include one connecting line to connect the two gas storage units, through which methane-rich biogas is fed from the first gas storage into the second gas storage unit on an as-needed basis, in order to enrich the gas stored there (see above).

Furthermore, for practical purposes, it must be emphasized that all valves previously described as valves that can be shut off are meant to be proportional valves, which can be set to any desired intermediate setting between a completely open and completely closed setting in order to adjust the respective throughput as desired.

The explicit process steps with which the plant is being run according the invention depend on various extant and plant-specific parameters and especially on the individual biomass. In that sense, the type of plant for the energetic use of the produced biogas determines what biogas methane concentrations can be used, and—considering the previously discussed values—taking into account future technical developments, the burning of biogas in a thermal power station, for example, is imaginable at methane concentrations much below 48%. Furthermore, the volume of the fermenter, the substrate fill level and the flushing air blower typically determine the volume of mixed gas and the methane concentration being produced during the flushing phase. This has an impact on the designed size or the degree of utilization of the second gas storage unit and on the duration of the (subsequent) use of the gas mixture, during which the second gas storage unit is being emptied, as well as on the amount of methane-rich gas with which the second gas storage unit has to be primed in order to always stay above the upper gas explosion limit.

### BRIEF DESCRIPTION OF THE DRAWINGS

The following further explains the present invention by using an application example, illustrated by a drawing. In it,

FIG. 1 shows the schematic of a plant featuring three fermenters for the methanation of biomass and the energetic use of the biogas produced, according the present invention.

FIG. 2 shows, with regard to the three fermenters of the plant illustrated in FIG. 1, the important phases of the respective cycle as well as the feeding of the two gas storage units and the gas removal from them.

### DETAILED DESCRIPTION

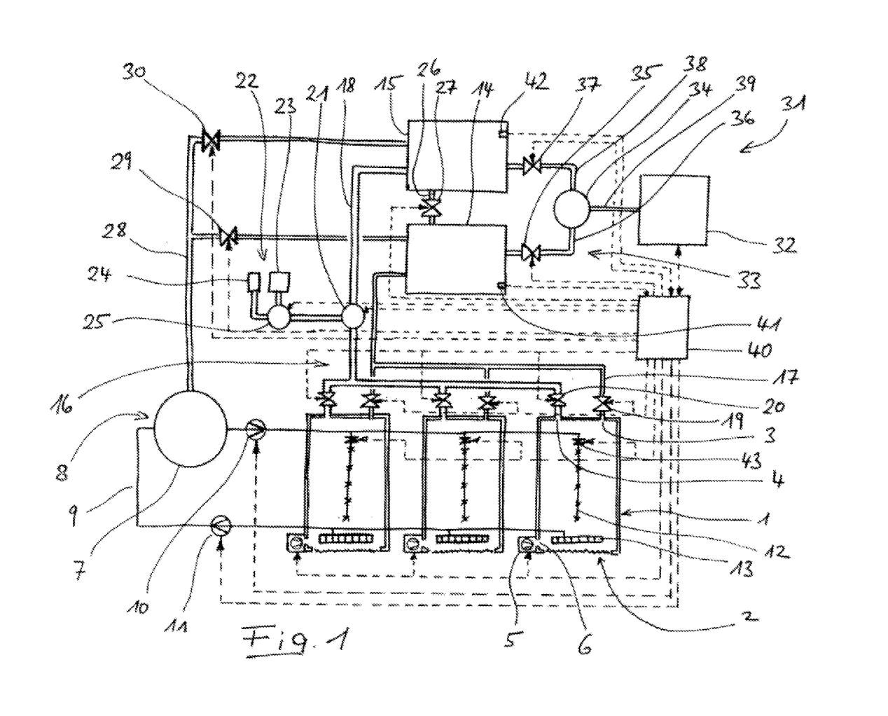

The plant shown in FIG. 1 for the methanation of biomass and energetic use of the biogas produced includes three identically built, garage-shaped fermenters 1. Each of the fermenters 1 features a loading opening 2 that can be closed in an airtight manner, a first gas outlet 3, a second gas outlet 4, as well as a flushing air blower 5 that is connected to a flushing air inlet 6. Furthermore, the plant includes a percolate system, including a percolate container 7, which is also a percolate fermenter 8, and it also features a percolate cycle. The percolate cycle shows percolate lines 9, pumps 10 and 11, as well as, for each fermenter 1, an irrigation strand 12—each controlled by a shut-off valve—and a drip collection channel 13.

Furthermore, two gas storage units are indicated, namely the first gas storage unit 14 and a second gas storage unit 15, each of which include at least one gas tank of standard construction.

The fermenters 1 are connected to the two gas storage units 14 and 15 via a network of gas lines 16. The latter includes a first gas line 17 and a second gas line 18, where the first gas line 17 (branched accordingly) connects the first gas storage unit 14 via one shut-off valve 19 each to the corresponding first gas outlet 3 of each fermenter 1, and the second gas line 18 (also branched accordingly) connects the second gas storage unit 15 via one shut-off valve 20 each to the corresponding gas outlet 4 of each fermenter 1.

In the second gas line 18, a controllable directional valve 21 is specified, which is used to optionally feed the gas leaving one or several fermenters 1 through the second gas outlet 4 into an off-gas system 22, rather than feeding the gas into the second gas storage 15. This off-gas system 22 includes a biofilter 23 and a gas torch 24, which can optionally be utilized via another controllable directional valve 25.

The first gas storage unit 14 and the second gas storage 15 are connected with a connection line 26 that has an installed shut-off valve, where in the connection line 26, a blower or a compressor—not shown—could also be specified under certain circumstances, in order to transport biogas from the first gas storage unit 14 into the second gas storage unit 15. Furthermore, the percolate fermenter 8 is connected to the first gas storage unit as well as to the second gas storage unit via a gas line 28 (branched accordingly), where a shut-off valve 29 or 30 is built into each of the two strands.

The plant 31 for the energetic use of the biogas produced includes a thermal power station 32, which is connected to a line network for usable gas 33 as part of the gas lines network 16, and to the first gas storage unit 14 as well as to the second gas storage unit 15. The line network for usable gas 33 features a gas mixer 34, into which lead—connected via a shut-off valve 35—a usable gas strand 36 from the first gas storage unit 14, and—via a shut-off valve 37—a usable gas strand 38 connected to a second gas storage unit 15. From the gas mixer 34, the usable gas is fed into the thermal power station 32 via the usable gas line 39. The respective operation of valves 35 and 37 allows for the optional connection of only the first gas storage unit 14, or of the first gas storage unit 14 and the second gas storage unit 15 simultaneously to the plant 31 for the energetic use of biogas via flow technology.

The system control unit 40 controls all controllable valves and shut-off valves, as well as pumps 10 and 11, the flushing blower 5 and the thermal power station 32. The system control unit 40 also uses current operating data from the thermal power station 32, and, in particular, the current operating data supplied by sensors 41 and 42 about the methane content of gases present in the first gas storage unit 14 or the second gas storage unit 15, the latter in particular with regard to the aforementioned enrichment of lean gas fed into the second gas storage unit 15 with methane-rich biogas from the fermenter 1 or from the percolate fermenter 8 in order to rule out the danger of an explosion. Particularly with regard to efficiency aspects, the minimization of emissions and the maximation of plant safety, including protection from explosions, various determined gas quality and volume stream measurements from gas line 17, 18 and 28, for example, have to be considered in the system control unit 40.

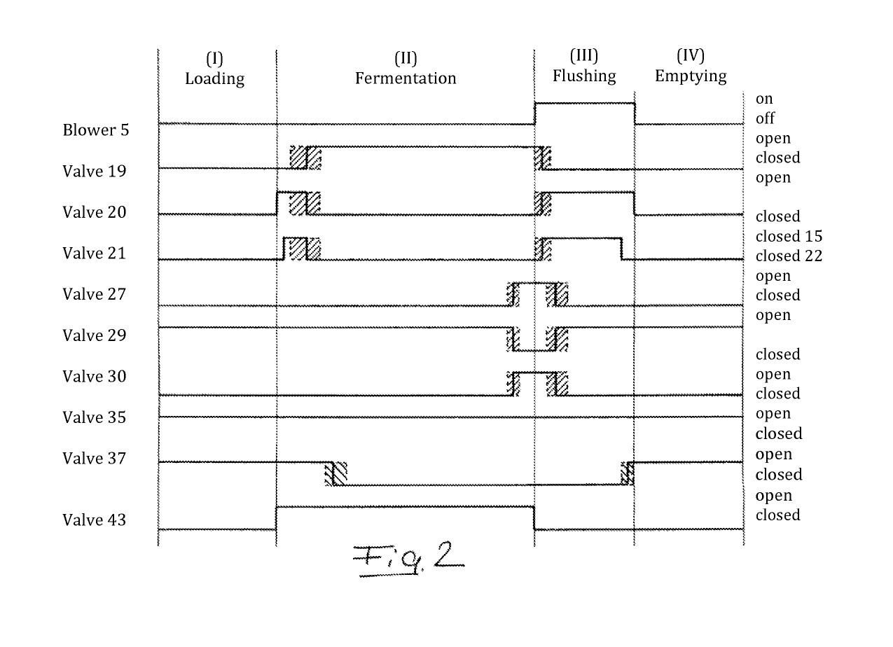

Using one of the three fermenters 1 from the plant illustrated in FIG. 1, FIG. 2 shows, as an example, the processes during a complete operation cycle—not to scale in terms of time relationships. The operation cycle is made up of roughly four phases, namely the feeding of biomass into the fermenter (part I), the fermentation phase (part II), the flushing phase (part III), and the emptying phase (part IV). The indicated positions of the various valves show, on the one hand, the feeding of the two gas storage units 14 and 15, changing in phases, from the respective fermenter 1 and the percolate fermenter 8, as well as, on the other hand, the removal of gas from the two gas storage units 14 and 15—also changing in phases—for energetic use in the thermal power station 32.

FIG. 2 illustrates that the following processes are being adhered to in the application example—where typical bandwidths in which the system control reacts to changing constraints are shown by shading areas for the opening and closing of valves:
During the feeding of the fermenter (phase I), both gas outlets 3 and 4 are closed using the corresponding valves 19 and 20. During this phase, the thermal power station 32 burns a mixed gas (methane content between about 48% and 60%), which is being produced in the gas mixer 34 by mixing gas removed from the first gas storage unit 14 (through the opened valve 35) and the second gas storage unit 15 (through the opened valve 37). During this phase, the percolate fermenter 8 feeds methane-rich biogas into the first gas storage unit 14.
During a more or less extended first part of the fermentation process (phase II), gas with very low methane content produced in fermenter 1, which in the beginning includes residual air, can be directed first to the biofilter 23 (through valves 20 and 21, adjusted accordingly), and subsequently gas relatively low in methane content (after adjusting valve 21) can be fed into the second gas storage unit. During this time period (fermentation start-up phase), mixed gas continues to be burned in the thermal power station 32. The percolate fermenter continues to feed methane-rich biogas into the first gas storage unit 14 during this time period.
During a second part of the fermentation phase (normal operation), with valves 19 and 20 being adjusted simultaneously in the beginning, methane-rich biogas produced in fermenter 1 is fed into the first gas storage unit 14 (through the opened valve 19, with valve 20 closed). During this phase, the thermal power station continues to burn mixed gas, doing so until the second gas storage unit 15 is empty, before burning exclusively biogas removed from the first gas storage unit 14. During this time, the percolate fermenter continues to feed methane-rich biogas into the first gas storage unit 14.
Towards the end of the fermentation phase, valves 29 and 30 are being (simultaneously) adjusted in such a way that methane-rich biogas from the percolate fermenter 8 is fed into the second gas storage unit 15, where, alternatively or in addition, methane-rich biogas from the first gas storage unit 14 can also be fed into the second gas storage unit 15—through the connecting line 26 with the opened valve 27.
During the first flushing phase (phase III), typically relatively methane-rich mixed gas leaving the fermenter 1 can be fed into the first gas storage unit 14 or the second gas storage unit 15, depending on concrete parameters regarding the prevailing gas volume and gas quality. During the following second part of the flushing phase, at the start of which valves 19 and 20 are adjusted simultaneously, mixed gas relatively low in methane content leaving the fermenter 1 is fed into the second gas storage unit 15.
During these two time periods, the thermal power plant continues to burn biogas removed from the first gas storage unit 14. The duration of the continued feeding of methane-rich primer gas at the beginning of the flushing phase into the second gas storage unit 15 from the percolate fermenter 8 (through the open valve 30) and/or the first gas storage unit 14 (through the open valve 27) depends on the estimated need of methane-rich biogas, in order to always keep the methane content of the gas in the second gas storage unit 15 above the upper explosion limit. Afterwards, the valves 29 and 30 are manipulated again so that from then on, methane-rich biogas from the percolate fermenter 8 is fed into the first gas storage unit 14 again. The thermal power plant continues to burn biogas removed from the first gas storage unit 14 during this time period as well. During a third period of the flushing phase, the mixed gas with very low methane content removed from fermenter 1 (with a methane content below the lower explosion limit) is cleaned via the biofilter 23 and released into the environment, with the valve 21 adjusted for this purpose. During this comparatively short phase, depending on prevailing operating conditions, only gas from the first gas storage unit 14 or, after adjustment of valve 37, mixed gas can already be burned in the thermal power plant 32. During the emptying phase, both gas outlets 3 and 4 are closed off using the corresponding valves 19 and 20. During this phase, the thermal power station 32 is burning a mixed gas, which is produced inside the gas mixer 34 by mixing gas removed from the first gas storage unit 14 and gas removed from the second gas storage unit 15.

We claim:

1. A method for the methanation of biomass and the energetic use of the biogas obtained in a plant featuring at least one fermenter (1) operating in batch processing mode, where in at least one fermenter the following sequence of steps is cyclically repeated:
a) Filling the fermenter (1) with biomass through a loading hatch (2) open to the environment,
b) airtight closing the loading hatch (2),
c) feeding methane-rich biogas produced during the fermentation phase (II), at least temporarily, into a first gas storage unit (14),
d) flushing the fermenter (1) with flushing air, and, at least temporarily, feeding the mixed gas with low methane content, which leaves the fermenter during the flushing phase (III), into a second gas storage unit (15),
e) opening the loading hatch (2), and
f) removing the digestate from the fermenter (1), where gas is removed from the second gas storage (15) unit and is mixed with biogas removed from the first gas storage (14), and the mixed gas produced in this way is subsequently used energetically.

2. Method according to claim 1, wherein during the first time period of the flushing phase (III), the mixed gas leaving the fermenter (1) is fed into the first gas storage unit (14).

3. Method according to claim 1, wherein during a last period of the flushing phase (III), the mixed gas leaving the fermenter (1) is released into the environment via a biofilter (23), or is burned using a torch (24).

4. Method according to claim 1, wherein the production and energetic use of mixed gas occurs intermittently.

5. Method according to claim 4, wherein the production and energetic use of mixed gas occurs during the change in biomass (Steps f and a), which is determined by the emptying phase (IV) and the loading phase (I), and the first time period (fermentation start-up phase) of the subsequent fermentation phase (II).

6. Method according to claim 5, wherein the second gas storage unit (15) is mostly emptied during the fermentation start-up phase.

7. Method according to claim 1, wherein after closing the loading opening (2), biogas with low methane content produced during a first time period (fermentation start-up phase) of the fermentation phase (II) is fed into the second gas storage unit (15).

8. Method according to claim 1, wherein the phases of feeding mixed gas with low methane content into the second gas storage unit (15) are shorter than the phases of removing gas from the second gas storage unit (15) for energetic use of the gas.

9. Method according to claim 1, wherein the methane concentration in the second gas storage unit (15) is always kept above the value of the upper explosion limit in air by adding methane-rich biogas.

10. Method according to claim 9, wherein pourable biomass is methanized by irrigating it with a percolate removed from a percolate fermenter (8), where methane-rich biogas fed into the second gas storage unit (15) is removed from the percolate fermenter.

11. Method according to claim 9, wherein the methane-rich biogas leaving the fermenter (1) is intermittently fed into the second gas storage unit (15), where the feeding of methane-rich biogas into the second gas storage unit (15) occurs before the flushing phase (III) and/or during the fermentation phase (II).

12. Method according to claim 9, wherein the methane-rich biogas fed into the second gas storage unit (15) is removed from the first gas storage unit (14) and transported to the second gas storage unit via a connecting line (26) that connects the two gas storage units.

13. Method according to claim 1, wherein the duration of the flushing phase (III) is 0.2 to 1.0%, preferably 0.3 to 0.8% of the cycle duration.

14. Method according to claim 1 wherein the rate of gas exchange during the flushing of the fermenter (1) with flushing air is between 1/h and 10/h, preferably between 2/h and 6/h, with regard to the gas volume inside the fermenter space.

15. Plant for the methanation of biomass and energetic use of the biogas produced according to the process in claim 1, including at least one loading opening (2) that can be closed in an airtight manner, at least one gas outlet (3, 4) as well as a fermenter featuring a flushing air inlet (6), at least one flushing air blower (5), a first gas storage unit (14) and a second gas storage unit (15), a gas line network (16) featuring several gas control valves (19, 20, 21, 35, 37), which connects at least one fermenter (1) with the first and the second gas storage unit (14, 15), a plant (31) for the energetic use of biogas, and a system control unit (40) that manipulates the gas control valves, where the gas control valves (19, 20, 21, 35, 37) can be manipulated by the system control unit (40) in such a way that the a least one gas outlet (3, 4) can optionally be connected to the first gas storage unit (14) or the second gas storage unit (15), and that optionally only the first gas storage unit (14) or simultaneously the first as well as the second gas storage unit (14, 15) can be connected to the plant (31) for the energetic use of biogas via flow technology.
